SEQUENCE LISTING (73 sequences)

SEQ ID NO: 1 - protein, 116 aa, synthetic construct
QVQLQQSGAELVKPGASVKLSCKASGYTFTEYTLQWVKQRSGQGLEWIGWFFPGSGSIKYNEKFKDKATLTADKSSNTVYMELSRLTSEDSAVYFCARHERGFAYWGQGTLVTVST

SEQ ID NO: 2 - protein, 116 aa, synthetic construct
QVQLVQSGAEVKKPGASVKVSCKASGYTFTEYTMHWVRQAPGQRLEWMGWFFPGSGSIKYSQKFQGRVTITRDTSASTAYMELSSLRSEDTAVYYCARHERGFAYWGQGTLVTVSS

SEQ ID NO: 3 - protein, 107 aa, synthetic construct
QIVLTQSPAIMSASLGEEITLTCSASSSVSYMHWYQQKSGTSPKLLIYSTSNLASGVPSRFSGSGSGTFYSLTISSVEAEDAADYHCHQWSSYPWTFGGGTKLEIKR

SEQ ID NO: 4 - protein, 108 aa, synthetic construct
DIVLTQSPASLAVSPGQRATITCRASASSSVSYIHWYQQKPGQPPKLLIYSTSNKDTGVPARFSGSGSGTDFTLTINPVEANDTANYYCHQWSSYPWTFGQGTKVEIK

SEQ ID NO: 5 - protein, 119 aa, synthetic construct
EVQLQQSGPELVKPGASVKISCKTSGYTFTEYTIHWVKQSHGKSLEWIGGINPNNGGTSYNQKFKGKATLTVDKSSSTPYMELRSLTSEDSAVYYCARLVLRYWYFDVWGAGTTVTVSS

SEQ ID NO: 6 - protein, 119 aa, synthetic construct
QVQLVQSGAEVKKPGASVKVSCKVSGYTFTEYTMHWVRQAPGKGLEWMGGINPNNGGTIYAQKFQGRVTMTEDTSTDTAYMELSSLRSEDTAVYYCARLVLRYWYFDVWGRGTLVTVSS

SEQ ID NO: 7 - protein, 106 aa, synthetic construct
QIVLTQSPAIMSASPGEKVTMTCSASSNVIYMHWYQQKSGTSPKRWLYDTSKLASGVPARFSGSGSGTSYSLTISSMEAEDAATYYCQQWSSNPLTFGAGTKLELK

SEQ ID NO: 8 - protein, 108 aa, synthetic construct
DIQMTQSPSSLSASVGDRVTITCRASASSNVIYLAWYQQKPGKAPKLLLYDTSRLESGVPSRFSGSGSGTDYTLTISSLQPEDFATYYCQQWSSNPLTFGGGTKVEIK

SEQ ID NO: 9 - protein, 119 aa, synthetic construct
QVQLQQSGPELVKPGTSVRISCKASGYTFTSYYIHWVKQRPGQGLEWIGWIYPENVNTKYNEKFKGRATLTADKSSNTAYMQLSSLTSEDSAVYFCARDSYGSYFFDYWGQGTTLTVSS

SEQ ID NO: 10 - protein, 119 aa, synthetic construct
QVQLVQSGAEVKKPGASVKVSCKASGYTFTSYYMHWVRQAPGQGLEWMGWIYPENVNTNYAQKFQGRVTMTRDTSISTAYMELSRLRSDDTAVYYCARDSYGSYFFDYWGQGTLVTVSS

SEQ ID NO: 11 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAMYYCTQSFILRTFGGGTKLEIK

SEQ ID NO: 12 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 13 - protein, 120 aa, synthetic construct
EVKLVESGGGLVKPGGSLKLSCAASGFTFSTYTMSWVRQTPAKSLEWVATINSDGYNIYYSDSMKGRFTISRDNARYTLYLQMSSLRSEDTAMYYCVRCSYYSYDYFDYWGQGTTLTVSS

SEQ ID NO: 14 - protein, 120 aa, synthetic construct
EVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSS

SEQ ID NO: 15 - protein, 107 aa, synthetic construct
DIVLTQSPASLSASVGETVTITCRASENIDNYLAWYQQKQGKSPQLLVYAATNLADGMPSRFSGSGSGTQYSLKINSLQSEDVARYFCQHYYSNQLTFGAGTKLELK

SEQ ID NO: 16 - protein, 107 aa, synthetic construct
DIQMTQSPSSLSASVGDRVTITCRASENIDNYLAWYQQKQGKVPKLLIYAATNLADGMPSRFSGSGSGTDFTLTISSLQPEDVATYYCQHYYSNQLTFGQGTKLEIK

SEQ ID NO: 17 - protein, 120 aa, synthetic construct
EVILVESGGDLVKPGGSLKLSCAASGFTFSRSTMSWVRQTPEKRLEWVATISGGGLNTYFPDSVKGRFTISRDNAKNTLYLHMSSLRSEDTAVYYCTRNSNSYYFALDYWGQGTSVTVSS

SEQ ID NO: 18 - protein, 120 aa, synthetic construct
EVQLVESGGGLVQPGGSLRLSCAASGFTFSRSTMSWVRQAPGKGLEWVATISGGGLNTYFPDSVKGRFTISRDNSKNTLYLQMNSLRAEDTAVYYCTRNSNSYYFALDYWGQGTLVTVSS

SEQ ID NO: 19 - protein, 111 aa, synthetic construct
DIVLTQSPASLAVSLGQRATISCRASESVDNSGVSFMNWFQQKPGQSPKLLIYIASNQVSGVPARFSGSGSGTDFSLNIHPMEEDDTAMYFCQQIKEVPWTFGGGTKLEIK

SEQ ID NO: 20 - protein, 111 aa, synthetic construct
EIVLTQSPATLSLSPGERATLSCRASESVDNSGVSFMNWYQQKPGQAPRLLIYIASNQVSGIPARFSGSGSGTDFTLTISSLEPEDFAVYYCQQIKEVPWTFGGGTKVEIK

SEQ ID NO: 21 - protein, 716 aa, synthetic construct
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPAGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 22 - protein, 234 aa, synthetic construct
MRLPAQLLGLLMLWVSGSSGDIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 720 aa, synthetic construct
MEFGLSWVFLVALLRGVQCEVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPAGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 24 - protein, 234 aa, synthetic construct
MRLPAQLLGLLMLWVSGSSGDIQMTQSPSSLSASVGDRVTITCRASENIDNYLAWYQQKQGKVPKLLIYAATNLADGMPSRFSGSGSGTDFTLTISSLQPEDVATYYCQHYYSNQLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 718 aa, synthetic construct
MEFGLSWVFLVALLRGVQCEVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPAGGGGSEVQLVESGGGLVQPGGSLRLSCAASGFTFSRSTMSWVRQAPGKGLEWVATISGGGLNTYFPDSVKGRFTISRDNSKNTLYLQMNSLRAEDTAVYYCTRNSNSYYFALDYWGQGTLVTVSSGGGGSGGGGSGGGGSEIVLTQSPATLSLSPGERATLSCRASESVDNSGVSFMNWYQQKPGQAPRLLIYIASNQVSGIPARFSGSGSGTDFTLTISSLEPEDFAVYYCQQIKEVPWTFGGGTKVEIK

SEQ ID NO: 26 - protein, 234 aa, synthetic construct; identical to SEQ ID NO: 22

SEQ ID NO: 27 - protein, 610 aa, synthetic construct
GTATMRLPAQLLGLLMLWVSGSSGDIQMTQSPSSLSASVGDRVTITCQASQDISNYLNWYQQKPGKAPKLLIYYTSRLESGVPSRFSGSGSGTDYTLTISSLQPEDFATYYCQQGNTLPWTFGQGTKVEIKGAPGGGGTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLSPGKPAGGGGSEVQLLESGGGLVQPGGSLRLSCAASGFTFSSYAMSWVRQAPGKGLEWVAVIDSNGGSTYYPDTVKDRFTISRDNSKNTLYLQMNSLRAEDTAVYYCSSYTNLGAYWGQGTLVTVSAGGGGSGGGGSGGGGSDIQMTQSPSSLSASVGDRVTITCRASQDISGYLNWLQQKPGKAIKRLIYTTSTLDSGVPSRFSGSGSGTDFTLTISSLQPEDFATYYCLQYASSPFTFGGGTKVEIK

SEQ ID NO: 28 - protein, 619 aa, synthetic construct
MEFGLSWVFLVALLRGVQCQVQLVQSGAEVKKPGASVKVSCKASGYTFTEYTMHWVRQAPGQRLEWMGWFFPGSGSIKYSQKFQGRVTITRDTSASTAYMELSSLRSEDTAVYYCARHERGFAYWGQGTLVTVSSGGGGSGGGGSGGGGSDIVLTQSPASLAVSPGQRATITCRASASSSVSYIHWYQQKPGQPPKLLIYSTSNKDTGVPARFSGSGSGTDFTLTINPVEANDTANYYCHQWSSYPWTFGQGTKVEIKGGGGSQVQLVQSGAEVKKPGASVKVSCKASGYSFTDYTMNWVRQAPGQGLEWMGVINPNHGISSYAQKFQGRVTMTRDTSTSTVYMELSSLRSEDTAVYYCVRRKISYDYDEGYAMDYWGQGTLVTVSSGAPGGGGTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSKLTVDKSRWQEGNVFSCSVMHEALHNHYTQKSLSLSLGK

SEQ ID NO: 29 - protein, 244 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 30 - protein, 246 aa, synthetic construct
QVQLVQSGAEVKKPGASVKVSCKASGYTFTSYYMHWVRQAPGQGLEWMGWIYPENVNTNYAQKFQGRVTMTRDTSISTAYMELSRLRSDDTAVYYCARDSYGSYFFDYWGQGTLVTVSSGGGGSGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 31 - protein, 246 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKGGGGSGGGGSGGGGSQVQLVQSGAEVKKPGASVKVSCKASGYTFTSYYMHWVRQAPGQGLEWMGWIYPENVNTNYAQKFQGRVTMTRDTSISTAYMELSRLRSDDTAVYYCARDSYGSYFFDYWGQGTLVTVSS

SEQ ID NO: 32 - protein, 261 aa, synthetic construct
QVQLVQSGAEVKKPGASVKVSCKASGYTFTSYYMHWVRQAPGQGLEWMGWIYPENVNTNYAQKFQGRVTMTRDTSISTAYMELSRLRSDDTAVYYCARDSYGSYFFDYWGQGTLVTVSSGGGGSGGGGSGGGGSGGGGSGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 33 - protein, 722 aa, synthetic construct
MEFGLSWVFLVALLRGVQCEVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPAGGGGSQVQLVQSGAEVKKPGASVKVSCKASGYTFTSYYMHWVRQAPGQGLEWMGWIYPENVNTNYAQKFQGRVTMTRDTSISTAYMELSRLRSDDTAVYYCARDSYGSYFFDYWGQGTLVTVSSGGGGSGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 34 - protein, 722 aa, synthetic construct
MEFGLSWVFLVALLRGVQCEVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPAGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKGGGGSGGGGSGGGGSQVQLVQSGAEVKKPGASVKVSCKASGYTFTSYYMHWVRQAPGQGLEWMGWIYPENVNTNYAQKFQGRVTMTRDTSISTAYMELSRLRSDDTAVYYCARDSYGSYFFDYWGQGTLVTVSS

SEQ ID NO: 35 - protein, 737 aa, synthetic construct
MEFGLSWVFLVALLRGVQCEVQLVESGGGLVQPGGSLRLSCAASGFTFSTYTMSWVRQTPAKGLVWVSTINSDGYNIYYSDSMKGRFTISRDNAKYTLYLQMNSLRAEDTAMYYCARCSYYSYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPAGGGGSQVQLVQSGAEVKKPGASVKVSCKASGYTFTSYYMHWVRQAPGQGLEWMGWIYPENVNTNYAQKFQGRVTMTRDTSISTAYMELSRLRSDDTAVYYCARDSYGSYFFDYWGQGTLVTVSSGGGGSGGGGSGGGGSGGGGSGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 36 - protein, 8 aa, synthetic construct
GYTFTEYT

SEQ ID NO: 37 - protein, 8 aa, synthetic construct
FFPGSGSI

SEQ ID NO: 38 - protein, 9 aa, synthetic construct
ARHERGFAY

SEQ ID NO: 39 - protein, 7 aa, synthetic construct
ASSSVSY

SEQ ID NO: 40 - skipped

SEQ ID NO: 41 - protein, 9 aa, synthetic construct
HQWSSYPWT

SEQ ID NO: 42 - protein, 8 aa, synthetic construct
GYTFTEYT

SEQ ID NO: 43 - protein, 8 aa, synthetic construct
INPNNGGT

SEQ ID NO: 44 - protein, 12 aa, synthetic construct
ARLVLRYWYFDV

SEQ ID NO: 45 - protein, 7 aa, synthetic construct
ASSNVIY

SEQ ID NO: 46 - skipped

SEQ ID NO: 47 - protein, 9 aa, synthetic construct
QQWSSNPLT

SEQ ID NO: 48 - protein, 8 aa, synthetic construct
GYTFTSYY

SEQ ID NO: 49 - protein, 8 aa, synthetic construct
IYPENVNT

SEQ ID NO: 50 - protein, 12 aa, synthetic construct
ARDSYGSYFFDY

SEQ ID NO: 51 - protein, 12 aa, synthetic construct
QSLLNSRTRKNY

SEQ ID NO: 52 - skipped

SEQ ID NO: 53 - protein, 8 aa, synthetic construct
TQSFILRT

SEQ ID NO: 54 - protein, 8 aa, synthetic construct
GFTFSTYT

SEQ ID NO: 55 - protein, 8 aa, synthetic construct
INSDGYNI

SEQ ID NO: 56 - protein, 13 aa, synthetic construct
ARCSYYSYDYFDY

SEQ ID NO: 57 - protein, 6 aa, synthetic construct
ENIDNY

SEQ ID NO: 58 - skipped

SEQ ID NO: 59 - protein, 9 aa, synthetic construct
QHYYSNQLT

SEQ ID NO: 60 - protein, 8 aa, synthetic construct
GFTFSRST

SEQ ID NO: 61 - protein, 20 aa, synthetic construct
ISGGGLNTYFPDSVKGRFTI

SEQ ID NO: 62 - protein, 13 aa, synthetic construct
TRNSNSYYFALDY

SEQ ID NO: 63 - protein, 10 aa, synthetic construct
ESVDNSGVSF

SEQ ID NO: 64 - skipped

SEQ ID NO: 65 - protein, 9 aa, synthetic construct
QQIKEVPWT

SEQ ID NO: 66 - protein, 116 aa, synthetic construct
EVQLVESGGGLVQPGGSLRLSCAASGFTFSSYAMSWVRQTPGKGLEWVAVIDSNGGSTYYPDTVKDRFTISRDNSKNTLYLQMNSLRAEDTAVYYCSSYTNLGAYWGQGTLVTVSA

SEQ ID NO: 67 - protein, 107 aa, synthetic construct
DIQMTQSPSSLSASVGDRVTITCRASQDISGYLNWLQQKPGGAIKRLIYTTSTLDSGVPKRFSGSGSGTDFTLTISSLQSEDFATYYCLQYASSPFTFGGGTKVEIK

SEQ ID NO: 68 - protein, 8 aa, synthetic construct
GFTFSSYA

SEQ ID NO: 69 - protein, 8 aa, synthetic construct
IDSNGGST

SEQ ID NO: 70 - protein, 9 aa, synthetic construct
SSYTNLGAY

SEQ ID NO: 71 - protein, 6 aa, synthetic construct
QDISGY

SEQ ID NO: 72 - skipped

SEQ ID NO: 73 - protein, 9 aa, synthetic construct
LQYASSPFT

(19) United States
(12) Patent Application Publication
(10) Pub. No.: US 2025/0034273 A1
(43) Pub. Date: Jan. 30, 2025

(54) COMPOSITION OF MULTISPECIFIC ANTIBODIES TARGETING CADHERIN-17-EXPRESSING TUMORS AND METHOD OF MAKING AND USING THEREOF

(71) Applicant: ARBELE Limited, Shatin, N.T. (HK)

(72) Inventors: John Moonching LUK, Bellevue, WA (US); Kwong Fai WONG, Tai Wai, N.T. (HK); Chu Qiao CHOW, Shatin (HK)

(21) Appl. No.: 18/696,919

(22) PCT Filed: Oct. 8, 2022
(86) PCT No.: PCT/US2022/077827

§ 371 Date: Mar. 28, 2024

Related U.S. Application Data
(60) Provisional application No. 63/253,612, filed on Oct. 8, 2021.

(51) Int. Cl. C07K 16/30; C07K 16/28

(52) U.S. Cl. CPC C07K 16/30; C07K 16/2809; C07K 16/2818; C07K 16/2896; C07K 2317/24; C07K 2317/31; C07K 2317/55; C07K 2317/565; C07K 2317/622

(57) ABSTRACT

A bispecific antibody, comprising IgG domains having heavy chains and light chains, and two scFv domains being connected to C terminal of the heavy chains, wherein the IgG domains comprises Fab regions having the binding specificity to a first antigen selected from Trop2 and CDH17, wherein the scFv domains have the binding specificity to a second antigen selected from CD3 and PD1.

### CROSS REFERENCE TO RELATED APPLICATIONS

This application claims the benefit of the filing date of U.S. Provisional Application Ser. No. 63/253,612 filed Oct. 8, 2021, under 35 U.S.C. 119(e), the entire disclosures of which are incorporated by reference herein.

### TECHNICAL FIELD

The present disclosure generally relates to the technical field of cancer immunotherapy, and more particularly to composition of modified antibodies with multiple antigen binding specificities.

### BACKGROUND

Gastrointestinal cancer is the cancer that develops along the GI tract, also known as the digestive tract. Gastrointestinal (GI) cancer includes all cancers in the digestive tract organs such as the stomach, large and small intestine, pancreas, colon, liver, rectum, anus, and biliary system. GI cancers are leading causes of morbidity and mortality worldwide, of which colorectal carcinoma (CRC) alone represents approximately 10% of all cancer diagnosis and is the second leading cause of cancer deaths world-wide after lung cancer. A recent survey in China reveals that the cancer spectrum of China is changing, with a rapid increase incidence and burden of lung, breast, colorectal, and prostate cancer in addition to a high incidence and heavy burden of liver, stomach, esophageal, and cervical cancer. Liver and stomach cancers remain prevalent and among the most lethal forms of malignancies, which are believed attributable to infections of Hepatitis B virus and Helicobacter pylori, as well as liver toxins and other environmental pollutants and food contaminants. There are no effective therapies for most forms of GI cancers. New biomarkers and therapeutic targets are thus needed for drug development against these aggressive cancers. A proven molecular targeting agent that can eliminate or repress the growth of these cancers will have important clinical value and significant market impact. These tumors can be resected effectively by surgery if the diseases are diagnosed in early stages. Unfortunately, and very often, most of GI cancers are asymptomatic and detected at very advanced stages when presented in the clinic. Without effective treatment, these patients die shortly after the diagnosis or relapse after salvage therapies.

Antibody-based targeted immunotherapy is a promising treatment of various forms of cancer, such as leukemia and lymphoma. But the success of antibody therapy for treating solid tumors such as GI cancers seems to be limited. It may be due to the heterogenicity of solid tumors and the tumor microenvironment, which often manifest tumor escape, off-tumor on-target toxicity, or immunosuppressive tumor microenvironment. To overcome the potential weakness and enhance the efficacy of antibody therapy in cancer treatment, a combination of target specificities may be explored. Antibody biologics may be a bi-specific or a tri-specific antibody targeting multiple tumor-associated antigens (TAA) or immune effector cell markers to achieve a more potent and specific response. Depending on the mechanism of action, a multi-specific antibody provides solutions to the issues. In the case of tumor escape, the antibody biologics may target multiple TAA such that, with loss of a single antigen on a tumor cell, the antibody binding still takes place and the anti-tumor activity remains effective. In the case of off-tumor on-target toxicity, instead of binding to a single TAA with high affinity, the binding to multiple TAAs with moderate affinity increases the selective avidity to the tumor cell relative to normal cells. Finally, targeting multiple immune checkpoint inhibitors, such as PD1, Tigit, and CTLA4, may help antagonize the immunosuppressive tumor microenvironment.

Cadherin-17 (CDH17) is a prominent cancer biomarker characterized by its overexpression in liver and stomach cancers but not in normal tissues. CDH17 is highly expressed in metastatic cancers. Anti-CDH17 monoclonal antibody displays the growth inhibitory effect on liver and stomach tumour cells, and the blockage of CDH17 expression and functions can markedly reduce lung metastasis of hepatocellular carcinoma (HCC). These observations indicate that humanized anti-CDH17 antibodies may be developed as target therapeutics for treating cancer patients with indication of CDH17 biomarker in tumour tissues and/or in serum samples.

While antibody drug conjugates are promising as an antibody therapy, multi-specific antibody therapeutics take advantage of immune responses to cancer and activate T cell-mediated cytotoxicity to cancer cells.

### SUMMARY

The following summary is illustrative only and is not intended to be in anyway limiting. In addition to the illustrative aspects, embodiments, and features described above, further aspects, embodiments, and features will become apparent by reference to the drawings and the following detailed description.

The application provides, among others, multi-specific antibodies, antibody-like proteins, and their derivative, fragments such as binding domains thereof, pharmaceutical compositions containing these antibodies or antibody-like proteins or binding domains, methods of making these antibodies or antibody-like proteins or binding domains, and methods of using antibodies or antibody-like proteins for treatment or diagnosis applications.

In one aspect, the application provides multi-specific antibodies and their derivatives or fragments thereof. In one embodiment, the antibody may include binding moieties to tumor antigens and effector cell antigens to generate T cell engagers.

In one embodiment, the application provides a multi-specific antibody comprising a heavy chain and a light chain. The heavy chain comprises of a first binding moiety selected from F(ab)2 and scFv, a CH1 domain, a hinge, and CH2 domain, and a second binding moiety as a scFv; the light chain comprises of a VL and CL domain.

In one embodiment, the application provides a bispecific antibody. In one embodiment, the bispecific antibody includes IgG domains having heavy chains and light chains, and two scFv domains being connected to C terminal of the heavy chains. The IgG domains comprises Fab regions having the binding specificity to a first antigen. The scFv domains have the binding specificity to a second antigen.

In one embodiment, the first antigen is selected from Trop2 and CDH17. In one embodiment, the first antigen is Trop2. In one embodiment, the first antigen is CDH17.

In one embodiment, the second antigen is selected from CD3 and PD1. In one embodiment, the second antigen is CD3. In one embodiment, the second antigen is PD1.

In one embodiment, the Fab domain comprises an amino acid sequence having at least 70%, 80%, 85%, 90%, 95%, 98%, or 99% of sequence identity to an amino acid sequence selected from SEQ ID NO: 13, 14, 15, 16, 66, and 67.

In one embodiment, the scFv domain comprises an amino acid sequence having at least 70%, 80%, 85%, 90%, 95%, 98%, or 99% of sequence identity to an amino acid sequence selected from SEQ ID NO: 29, 30, 31, and 32.

In one embodiment, the bispecific antibody has 3 heavy chain complementary determining regions (CDRs) of SEQ ID NO: 54, 55, 56 and 3 light chain CDRs of SEQ ID NO: 57, 58, 59. In one embodiment, the bispecific antibody has 3 CDRs of SEQ ID NO: 68, 69, 70 and 3 light chain CDRs of SEQ ID NO: 71, 72, 73.

In one embodiment, the bispecific antibody has 3 heavy chain CDRs of SEQ ID NO: 48, 49, 50 and 3 light chain CDRs of SEQ ID NO: 51, 52, 53. In one embodiment, the bispecific antibody has 3 heavy chain CDRs of SEQ ID NO: 60, 61, 62 and 3 light chain CDRs of SEQ ID NO: 63, 64, 65.

In one embodiment, the first antigen is Trop2, and the second antigen is CD3. In one embodiment, the antibody comprises an amino acid sequence having at least 70%, 80%, 85%, 90%, 95%, 98%, or 99% of sequence identity to an amino acid sequence selected from SEQ ID NO: 23, 24, 33, 34, 35.

In one embodiment, the first antigen is CDH17, and the second antigen is PD1. In one embodiment, the antibody comprises an amino acid sequence having at least 70%, 80%, 85%, 90%, 95%, 98%, or 99% of sequence identity to an amino acid sequence selected from SEQ ID NO: 25, 26.

In another aspect, the application provides tri-specific antibody like protein (TriAx-C), their derivatives or fragments thereof. In one embodiment, the tri-specific antibody like protein is a heterodimer.

In one embodiment, the tri-specific antibody like protein has a N terminus and a C terminus and comprises a first monomer and a second monomer. The first monomer comprises, from the N terminus to the C terminus, a first scFv domain having a binding specificity to a first antigen, a variable heavy chain, a first CH3 domain, and a second CH3 domain. The second monomer comprises, from the N terminus to the C terminus, a variable light chain, a CH2 domain, a third CH3 domain and a second scFv domain having a binding specificity to a second antigen. The variable heavy chain and the variable light chain form a Fab domain having a binding specificity to a third antigen.

In one embodiment, the first monomer and the second monomer are linked through one or more disulfide bonds. In one embodiment, the third CH3 domain and the second CH3 domain are configured to form a knob and hole structure.

In one embodiment, the first antigen is PDL1. In one embodiment, the second antigen is CDH17. In one embodiment, the third antigen is CD3. In one embodiment, the tri-specific antibody-like protein has a binding affinity to PDL1, CDH17 and CD3.

In one embodiment, the tri-specific antibody like protein of comprises 3 heavy chain CDRs of SEQ ID NO: 36, 37, 38 and 3 light chain CDRs of SEQ ID NO: 39, 40, 41. In one embodiment, the tri-specific antibody like protein comprises 3 heavy chain CDRs of SEQ ID NO: 68, 69, 70 and 3 light chain CDRs of SEQ ID NO: 71, 72, 73. In one embodiment, the tri-specific antibody like protein comprises 3 heavy chain CDRs of SEQ ID NO: 48, 49, 50 and 3 light chain CDRs of SEQ ID NO: 51, 52, 53.

In one embodiment, the tri-specific antibody like protein comprises an amino acid sequence having at least 70%, 80%, 85%, 90%, 95%, 98%, or 99% of sequence identity to an amino acid sequence selected from SEQ ID NO: 27, 28.

In a further aspect, the application provides antibodies having binding affinity to Tigit (Anti-Tigit mAb) and their derivatives or fragments thereof. In one embodiment, the antibody having a binding affinity to Tigit comprises 3 heavy chain CDRs of SEQ ID NO. 42, 43, 44 and 3 light chain CDRs of SEQ ID NO. 45, 46, 47. In one embodiment, the monoclonal antibody comprises an amino acid sequence having at least 70%, 80%, 85%, 90%, 95%, 98%, or 99% sequency identity a SEQ ID NO. 5, 6, 7, or 8.

The antibody disclosed herein may be a humanized antibody, a chimeric antibody, or a recombinant antibody, or an isolated monoclonal antibody.

In a further aspect, the application provides binding domains for antibodies or antibody-like proteins.

In one embodiment, the application provides Fab domains. In one embodiment, the Fab domain has 3 heavy chain CDRs of SEQ ID NO: 54, 55, 56 and 3 light chain CDRs of SEQ ID NO: 57, 58, 59. In one embodiment, the Fab domain has 3 heavy chain CDRs of SEQ ID NO: 68, 69, 70 and 3 light chain CDRs of SEQ ID NO: 71, 72, 73.

In one embodiment, the application provides scFv domains. In one embodiment, the scFv domain has 3 heavy chain CDRs of SEQ ID NO: 48, 49, 50 and 3 light chain CDRs of SEQ ID NO: 51, 52, 53. In one embodiment, the scFv domain has 3 heavy chain CDRs of SEQ ID NO: 60, 61, 62 and 3 light chain CDRs of SEQ ID NO: 63, 64, 65.

In one embodiment, the application provides a binding domain having an affinity to PDL1. In one embodiment, the binding domain comprises 3 heavy chain CDRs of SEQ ID NO: 36, 37, 38 and 3 light chain CDRs of SEQ ID NO: 39, 40, 41.

In one embodiment, the application provides a binding domain having an affinity to CDH17. In one embodiment, the binding domain comprises 3 heavy chain CDRs of SEQ ID NO: 68, 69, 70 and 3 light chain CDRs of SEQ ID NO: 71, 72, 73.

In one embodiment, the application provides a binding domain having an affinity to CD3. In one embodiment, the binding domain comprises 3 heavy chain CDRs of SEQ ID NO: 48, 49, 50 and 3 light chain CDRs of SEQ ID NO: 51, 52, 53.

In one embodiment, the application provides a binding domain having an affinity to Tigit. In one embodiment, the binding domain comprises 3 heavy chain CDRs of SEQ ID NO. 42, 43, 44 and 3 light chain CDRs of SEQ ID NO. 45, 46, 47.

In a further aspect, the application provides isolated nucleic acid sequence encoding the antibodies, antibody-like proteins, their binding domains, derivatives, or fragments thereof.

In a further aspect, the application provides expression vector comprising the isolated nucleic acid sequences disclosed herein.

In a further aspect, the application provides host cell comprising the isolated nucleic acid sequence disclosed herein. In one embodiment, the host cell comprises the expression vector disclosed herein. In one embodiment, the host cell may be a prokaryotic cell or a eukaryotic cell.

In a further aspect, the application provides method for producing the antibodies, antibody-like proteins, their binding domains, derivatives, or fragments thereof. In one embodiment, the method includes the step of culturing the host cell disclosed herein so that the antibodies, antibody-like proteins, their binding domains, derivatives, or fragments thereof is produced.

In a further aspect, the application provides immunoconjugates. In one embodiment, the immunoconjugate includes a cytotoxic agent. In one embodiment, the immunoconjugate includes an imaging agent. The cytotoxic agent or the imaging agent may be conjugated to the antibodies, antibody-like proteins, their binding domains, derivatives, or fragments thereof through a chemical linkage including such as a covalent bond, an amide bond, a peptide bond, an ether bond, or an ester bond.

In a further aspect, the application provides pharmaceutical composition, comprising a pharmaceutically acceptable carrier and the antibodies, antibody-like proteins, their binding domains, derivatives, fragments, or immunoconjugate as disclosed thereof. In one embodiment, the pharmaceutical composition may further comprise a radioisotope, a radionuclide, a toxin, a therapeutic agent, a chemotherapeutic agent or a combination thereof.

In a further aspect, the application provides methods for treating or preventing a cancer, an autoimmune disease, or an infectious disease in a subject, said method comprising administering to the subject a pharmaceutical composition disclosed herein. In one embodiment, the method of treating a subject with a cancer include the step of administering to the subject an effective amount of the bispecific antibody disclosed herein.

In one embodiment, the method of treating comprises co-administering to the subject an effective amount of a therapeutic agent. In one embodiment, the therapeutic agent comprises an antibody, a chemotherapy agent, an enzyme, or a combination thereof.

In one embodiment, the subject is a human.

In a further aspect, the application provides a solution comprising an effective concentration of the antibodies, antibody-like proteins, their binding domains, derivatives, fragments, or immunoconjugate as disclosed thereof. In one embodiment, the solution includes an effective concentration of bispecific antibody as disclosed herein. In one embodiment, the solution is blood plasma or a body fluid in or from the subject.

### BRIEF DESCRIPTION OF THE DRAWINGS

Embodiments according to the present disclosure may now be described with reference to the figures, in which like reference numerals denote like elements.

FIG. 1 depicts configurations of two bi-specific antibodies, ARB203 and ARB204; and a tri-specific antibody, TriAx-C;

FIG. 2 shows that anti-PDL1 (clone 2A6) binds to 293F cells expressing PDL1 (left) and blocks PD1-PDL1 binding (right);

FIG. 3 shows that anti-Tigit antibody (clone 4F11) binds to 293F cells expressing Tigit (left) and blocks CD155 binding to Tigit (right);

FIG. 4 depicts a bispecific antibody of anti-Trop2 (h8B5) and anti-CD3 (clone 3G8). (A) Different arrangements of h3G8 scFv impact its binding to human and monkey CD3. V2 and V3 maintained binding to both. (B) Affinity of V2 towards human and monkey CD3 is comparable. (C) T-cell mediated cytotoxicity on cancer cells between variants was compared. V1 had highly reduced killing effect while V2 was closest to clone UCHT1 in terms of EC50. (D) During T-cell mediated cytotoxicity, production of different cytokines was induced. Comparing to clone UCHT1, V2 had similar EC50 in inducing secretion of IL2 and IFNγ;

FIG. 5 shows ARB203, a bispecific antibody of anti-Trop2 (h8B5) and anti-CD3 (UCHT1). (A) ARB203 bound to Trop2-positive cell line (SW480) but not Trop2-negative cell line (A549). (B) Affinity of clone 8B5 towards human Trop2 was extremely high (K_{D}=1E⁻¹²M), which was beyond the limit of measurement of the Octet system. (C) ARB203 was very potent and could induce 100% killing of cancer cells at less than 1% receptor occupancy on both T cells and cancer cells; and

FIG. 6 shows ARB204, a CDH17-PD1 bispecific antibody, binds to HEK293F expressing PD1 (left) and shows strong cytotoxic effect on AsPC1 (right).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

To enable immunotherapeutics for more effective cancer treatment, especially solid tumors, a combination therapeutic that incorporates multiple target specificities and/or mechanisms of action may be used. A therapeutic that is a combination treatment such as that described here, is necessary to effectively treat cancer and to more frequently achieve a complete and durable response. Specifically, there is a need for a scaffold that possesses certain properties to create a combination therapeutic with advantageous mechanisms of action, manufacturing, pharmacokinetics, and low antigenic properties relative to approved bispecific antibodies. Bispecific antibodies that are based on a whole antibody may have a greater mass relative to the tri-specific antibodies described here. The conventional bispecific antibodies redirecting T-cells to eradicate cancer cells have limited efficacy because of suboptimal effector cell engagement. More efficient T-cell activation has been obtained with single-chain variable fragment (scFv) antibodies, notably Bispecific T-cell Engagers (BiTEs). While antibodies with a smaller mass, based on antibody fragments, may have greater tumor penetration, they often possess relatively poor pharmacokinetic properties, such as FDA-approved anti-CD19/CD3 bispecific BiTE antibody, blinatumomab (BLINCYTO®, Amgen) for patients with chemotherapy-resistant CD19+ B-cell acute lymphoblastic leukemia (B-ALL). Many BiTEs are in clinical testing in several solid tumors and hematologic malignancies, at least the clinical experience with blinatumomab has demonstrated that many patients fail BiTE therapy for poorly understood reasons despite target antigen expression on their cancer cells. All these BiTEs rely on CD3 signaling and anti-TAA without providing co-stimulation or inhibition of any immune checkpoint.

CDH17 is highly expressed in metastatic GI-cancers, and the blockage of CDH17 expression and functions can also markedly reduce lung metastasis of hepatocellular carcinoma (HCC). The anti-CDH17 monoclonal antibody and anti-CDH17/CD3 bispecific antibody (e.g., ARB202) have been developed and characterized, which display the growth inhibitory effect on liver and stomach tumor cells (see Applicant's application WO/2019/222428 and U.S. Pat. No. 11,207,419, incorporated herein by reference in their entirety). Like CDH17, TROP2 is a prominent cancer biomarker characterized by its overexpression in various forms of solid tumors, including stomach, colon, pancreatic, liver and liver. To maximize the benefit of antibody therapy targeting CDH17 to the patients with GI-cancers, additional binding specificity may be required.

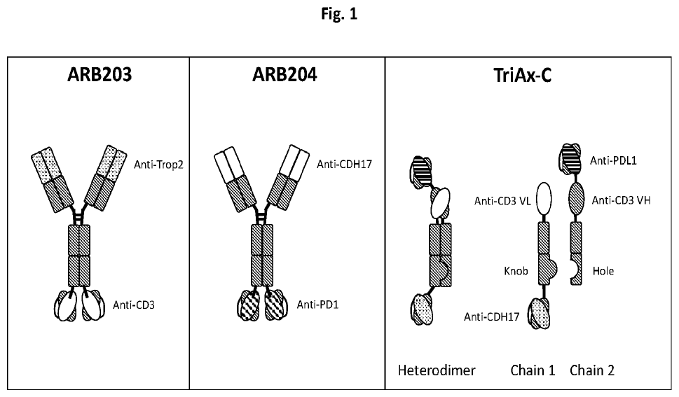

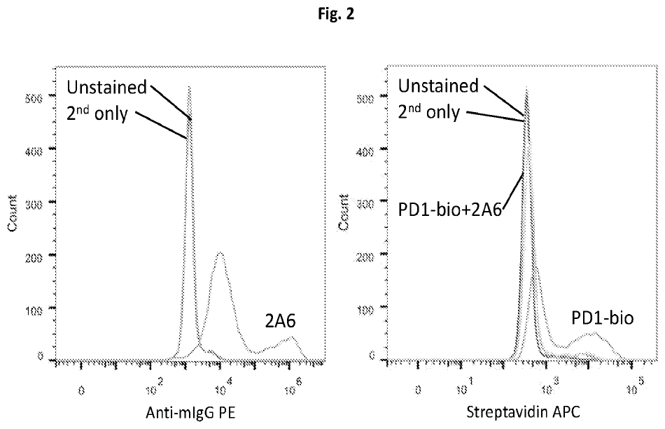

In one aspect, the application discloses bispecific antibodies having two binding specificities. Example bispecific antibodies include ARB203 (anti-Trop2/CD3) and ARB204 (anti-CDH17/PD1) (FIG. 1).

In one aspect, the application additional discloses trispecific antibodies or antibody-like proteins (in a TriAx-C format). In one embodiment, the tri-specific antibody like protein has the binding specificity to either PDL1 or TIGIT for inhibiting the immune checkpoint may be configured to add to the anti-CDH17/CD3 specificities (FIG. 1). TIGIT is an immune receptor present on some T cells and natural killer cells (NK) that could bind to CD155 on dendritic cells (DCs), macrophages, etc. with high affinity. Several clinical trials of TIGIT combination treatments show promise for combined TIGIT and PD-L1 co-blockade in patients with solid cancer.

The disclosed TriAx-C format is characterized by having the knobs-into-holes structure with no mutation in any constant domain. This configuration differs from many bispecific antibodies that possess both the knobs-into-holes and mutations within the constant domains of the Ig structure, which may contribute to an anti-drug antibody response.

The terms “a”, “an” and “the” as used herein are defined to mean “one or more” and include the plural unless the context is inappropriate.

The term “antibody” is used in the broadest sense and specifically covers single monoclonal antibodies (including agonist and antagonist antibodies), antibody compositions with polyepitopic specificity, as well as antibody fragments, such as Fab, F(ab′)2, and Fv, so long as they exhibit the desired biological activity. In some embodiments, the antibody may be monoclonal, chimeric, single chain, multi-specific, multi-effective, human, and humanized antibodies. Examples of active antibody fragments that bind to known antigens include Fab, F(ab′)2, scFv, and Fv fragments, as well as the products of a Fab immunoglobulin expression library and epitope-binding fragments of any of the antibodies and fragments mentioned above. In some embodiments, antibody may include immunoglobulin molecules and immunologically active portions of immunoglobulin molecules, i.e., molecules that contain a binding site that immunospecifically bind to an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain moiety is comprised of a light chain moiety variable domain (abbreviated as VL) and a light chain moiety constant domain. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the heavy and light chain there are binding regions that interacts with the antigen.

The term “multi-specific” antibody as used herein denotes an antibody that has at least two binding sites each having a binding affinity to an epitope of an antigen. The term “bi-specific, tri-specific, tetra-specific, or penta-specific” antibody as used herein denotes an antibody that has two, three, four, five, or six antigen-binding sites.

The term “humanized antibody” antibody refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art (see Queen et al., Proc. Natl Acad Sci USA, 1989; Hodgson et al., Bio/Technology, 1991). In one embodiment, the “humanized antibody” may be obtained by genetic engineering approach that enables production of affinity-matured humanlike polyclonal antibodies in large animals such as, for example, rabbits (see U.S. Pat. No. 7,129,084).

The term “antibody” or “antibody-like protein” may be used interchangeably in the application.

The term “antigen” refers to an entity or fragment thereof which can induce an immune response in an organism, particularly an animal, more particularly a mammal including a human. The term includes immunogens and regions thereof responsible for antigenicity or antigenic determinants.

The term “epitope”, also known as “antigenic determinant”, is the part of an antigen that is recognized by the immune system, specifically by antibodies, B cells, or T cells, and is the specific piece of the antigen to which an antibody binds.

The term “immunogenic” refers to substances which elicit or enhance the production of antibodies, T-cells, or other reactive immune cells directed against an immunogenic agent and contribute to an immune response in humans or animals. An immune response occurs when an individual produces sufficient antibodies, T-cells, and other reactive immune cells against administered immunogenic compositions of the present application to moderate or alleviate the disorder to be treated.

The term “tumor antigen” as used herein means an antigenic molecule produced in tumor cells. A tumor antigen may trigger an immune response in the host. In one embodiment, the tumor cells express tumor antigens, including without limitation, tumor-specific antigens (TSA), neoantigens, and tumor-associated antigens (TAA).

The term “specific binding to” or “specifically binds to” or “specific for” a particular antigen or an epitope as used herein means the binding that is measurably different from a non-specific interaction. Specific binding can be measured by determining binding of a molecule compared to binding of a control molecule, which generally is a molecule of similar structure that does not have binding activity. Specific binding can be determined by competition with a control molecule that is like the target. Specific binding for a particular antigen or an epitope can be exhibited by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹, alternatively at least about 10⁻¹⁰ M, at least about 10⁻¹¹ M, at least about 10⁻¹² M, or greater, where KD refers to a dissociation rate of a particular antibody-antigen interaction. In some embodiments, a multi-specific antibody that specifically binds to an antigen will have a KD that is 20-, 50-, 100-, 500-, 1000-, 5,000-, 10,000- or more times greater for a control molecule relative to the antigen or epitope. Also, specific binding for a particular antigen or an epitope can be exhibited by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The disclosure may be understood more readily by reference to the following detailed description of specific embodiments and examples included herein. Although the disclosure has been described with reference to specific details of certain embodiments thereof, it is not intended that such details should be regarded as limitations upon the scope of the disclosure. Indeed, various modifications of the disclosure in addition to those described herein will become apparent to those skilled in the art from the foregoing description and accompanying drawings. Such modifications are intended to fall within the scope of the appended claims.

### EXAMPLES

The present disclosure is further described with reference to the following examples. These examples are provided for purposes of illustration only and are not intended to be limiting unless otherwise specified. Those of skill in the art will readily recognize a variety of non-critical parameters that could be changed or modified to yield essentially the same or similar results.

### Example 1. Configurations of Multi-Specific Antibodies or Antibody-Like Proteins

As illustrated in FIG. 1, the bispecific antibodies, such as ARB203 and ARB204, may be constructed by using IgG4 Fc as its backbone structure. The hinge region is switched to its IgG1 counterpart as to prevent arms switching. With this platform, ARB203 was constructed as a bispecific anti-Trop2/CD3 antibody. ARB203 is bivalent to Trop2 via a Fab region and CD3 a scFv domain which is covalently linked to the C-terminus of its heavy chain. With this configuration, ARB203 may function as a T-cell engager bringing activated T-cells to Trop2-expressing tumor cells and elicit immune response against the cancer cells.

ARB204 is a bispecific anti-CDH17/PD1 antibody with IgG4 Fc as its backbone structure, as illustrated in FIG. 1. The hinge region is switched to its IgG1 counterpart as to prevent arms switching. ARB204 is bivalent to each of two binding targets, of which its Fab region binds to CDH17 and the covalently linked scFv domain binds PD1. With this configuration, ARB204 functions to blockade the immune checkpoint around CDH17-expressing tumor cells and elicit immune response to GI cancers.

Also illustrated in FIG. 1, the trispecific anti-CDH17/CD3/PDL1 antibody-like protein is an exemplary antibody-like protein in an asymmetrical TriAx-C format. These antibody-like protein may be characterized by having a knobs-in-holes substructure to ensure a correct pairing of the heterodimeric antibody. In this TriAx-C example, the variable light chain (VL) of anti-CD3 is fused to Fc-knob and anti-CDH17 scFv to form the Chain 1, whereas the anti-PDL1 scFv is fused to variable heavy chain (VH) of anti-CD3 and Fc-hole to form the Chain 2. A correct pairing of the two chains forms a heterodimeric Ig structure containing three binding specificities.

### Example 2. Antibodies and Binding Fragments Against CDH17, Trop2, PD1, PDL1, Tigit, and CD3

Each plasmid containing one antibody-coding sequence was transfected into HEK293 or CHO cells for mammalian cell expression. The antibody, antibody-like protein or fragment may be purified from the culture supernatant with protein-A affinity chromatography. The monoclonal antibodies specifically against PDL1 (2A6), Tigit (4F11), CD3 (3G8), Trop2 (8B5), and PD1 (1C8) were generated with hybridoma technology and humanized. The novel sequences encoding the heavy and light chain of each mouse and humanized monoclonal antibody are listed in Table 1.

To assess the function of each monoclonal antibody, humanized anti-PDL1 antibody (Clone 2A6, SEQ ID NO. 3, 4) was used as an example. As shown in FIG. 2, HEK293 cells expressing PDL1 was stained with biotinylated PD1 and visualized using streptavidin APC. The result of the histogram analysis shows that Clone 2A6 binds to HEK293 cells expressing PDL1 and blocks PD1-PDL1 interaction, which validates the anticipated binding specificity to PDL1 and blocking PD1-PDL1 interaction.

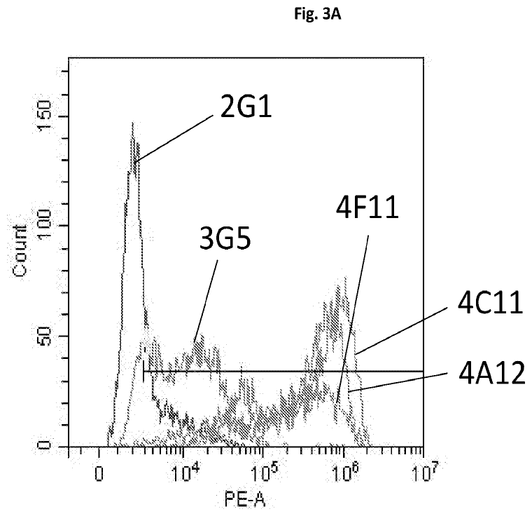

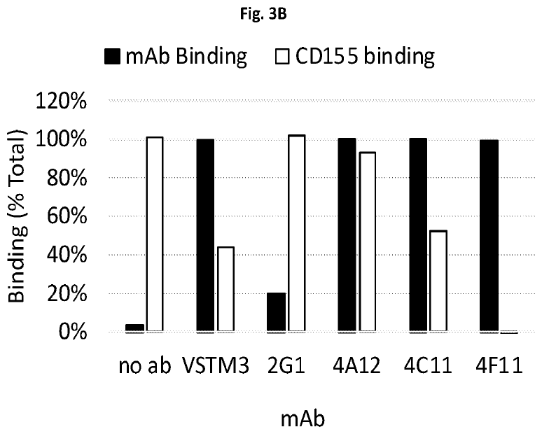

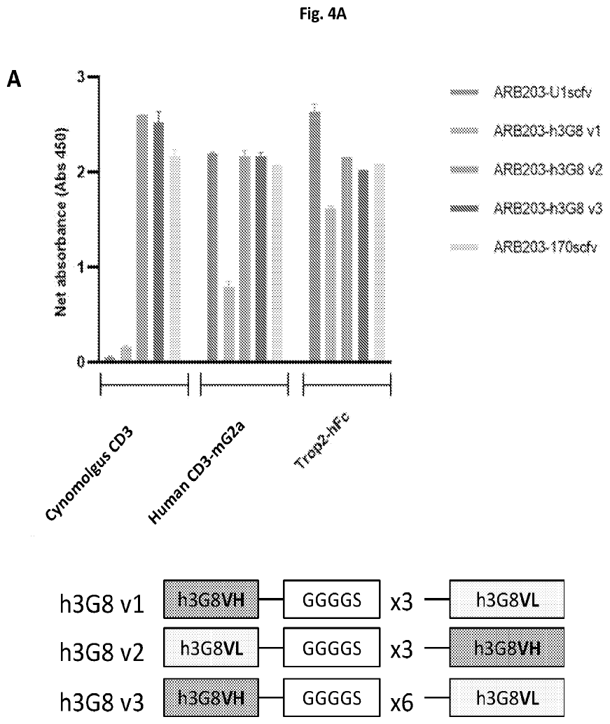

In another example, humanized anti-Tigit antibody or Clone 4F11 was chosen for characterizing its ability of blocking CD155 from binding to Tigit. As shown in FIG. 3, while CD155 can bind to the HEK293 cells engineered to express Tigit, Clone 4F11 was also capable of binding to Tigit-expressing HEK293 cells. These results demonstrate that Clone 4F11 comprises anti-Tigit binding domain capable of blocking the CD155-Tigit interaction.

### Example 3. Bispecific Antibodies

The plasmids containing bispecific antibody-coding sequences were transfected or co-transfected into HEK293 or CHO cells for mammalian cell expression. The antibody or fragment may be purified from the culture supernatant with protein-A affinity chromatography.

ARB203 is a bispecific antibody capable of binding to Trop2, a tumor-associated antigen, and CD3, a T-cell surface marker, such that it may act to engage T-cell dependent cytotoxicity to Trop2-expressing cancer cells. In the sequence listing, the VH/VL sequence of a panel of novel antibodies is given. With the amino acid sequences of each domain or binding fragment derived from the corresponding monoclonal antibodies, the full-length sequences of ARB203 were listed (SEQ ID NO. 23, 24).

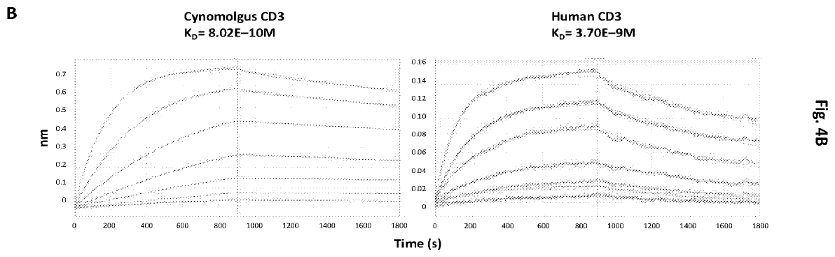

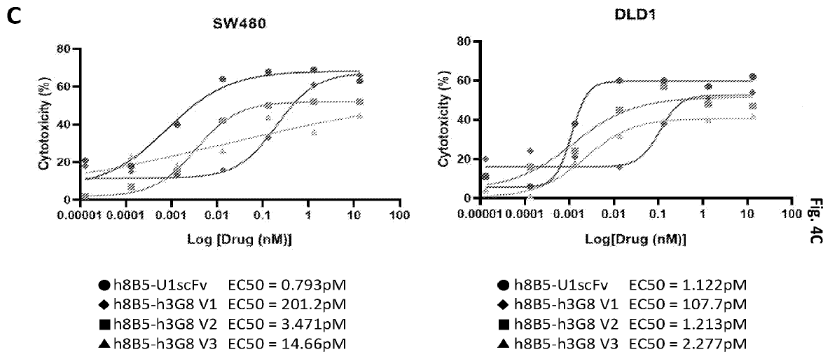

While the Trop2 binding domain of ARB203 was derived from the Fab region of an anti-Trop2 antibody (Clone 8B5), the CD3-binding scFv domain was derived from an anti-CD3 antibody (Clone UCHT1). To optimize CD3 binding, the novel anti-CD3 clone 3G8 was introduced to replace UCHT1. Three versions of 3G8scFv domains (V1 to V3) were tested with differences in the orientation and the length of Gly-Ser (GS) linker used to be covalently linked to ARB203 heavy chain. V1 is in VH-VL with 3×GS linker; V2 is in VL-VH with 3×GS linker; and V3 is in VH-VL with 6×GS linker. The Octet binding analysis revealed that both V2 and V3 were capable of binding to both human and monkey CD3 (FIG. 4A). In addition, the respective affinities were assessed for V2 (FIG. 4B). When incorporated as a T-cell engager into the bispecific antibody, ARB203 exerted potent killing and cytokine induction (FIGS. 4C&D). Of three variants, V2 had the most favorable cytotoxicity and cytokine profile, which was comparable to that of a known anti-CD3 clone, UCHT1.

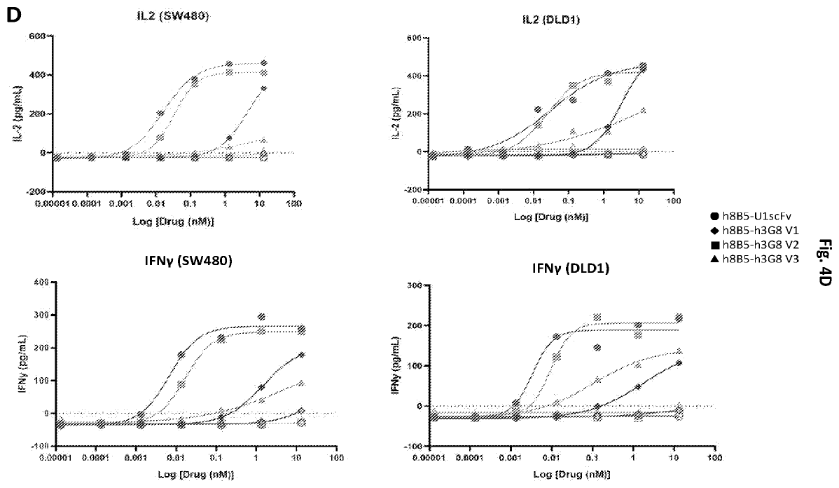

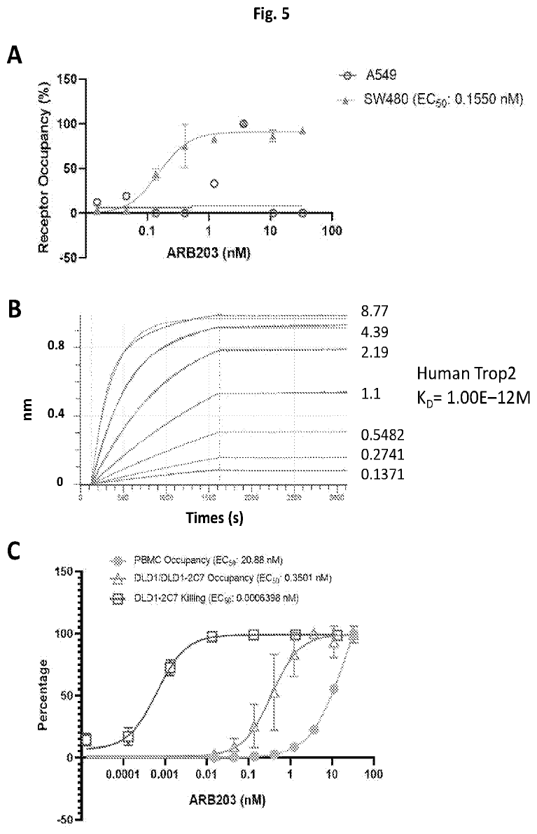

To assess its binding specificity to Trop2, ARB203 was characterized by its capability of binding to Trop2-expressing HEK293 cells and high affinity to Trop2 on the Octet platform (FIGS. 5B&C). Furthermore, ARB203 exerted potent cytotoxicity against cancer cell line and <1% receptor occupancy was required for 100% killing (FIG. 5D).

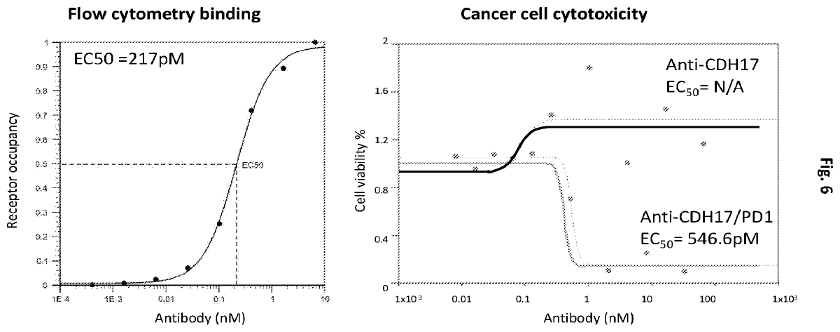

Like the approach for generating ARB203, ARB204 was created and expressed starting from an anti-PD1 antibody (Clone 1C8) scFv and incorporating the anti-CDH17 Fab domain, which was derived from Clone 10C12, into the IgG-scFv format. In this way, ARB204 differs from ARB203 in their mechanism of actions. ARB204 targets CDH17-expressing GI-cancer cells while inhibiting immune checkpoint by blocking the PD1-PDL1 mediated cross talk. Indeed, ARB204 bound to HKE293F expressing PD1 and showed potent cytotoxicity against AsPC1 pancreatic cell line (FIG. 6).

### Example 4. Trispecific Antibodies or Antibody-Like Proteins

The configuration of trispecific antibodies in a TriAx-C platform is unique, of which a combination of scFvs, split VH/VL, and the knob-hole Fc domain may be incorporated into Chain 1 and Chain 2 of a heterodimeric antibody structure as illustrated in FIG. 1. With this TriAx-C platform, any combination of specificities against TAA (e.g., CDH17, Trop2), T-cell engager (CD3), and immune checkpoint targets (e.g., PD1, PDL1, Tigit) can be configured to generate desirable trispecific antibodies for different indications.

TABLE 1
Sequence Listing

| SEQ | | Binding | |
|---|---|---|---|
| ID NO | Clone | specificity | Description |
| 1 | 2A6 | PDL1 | Mouse VH sequence, with 3 CDRs underlined |
| 2 | | | Humanized VH sequence, with 3 CDRs underlined |
| 3 | | | Mouse VL sequence, with 3 CDRs underlined |
| 4 | | | Humanized VL sequence, with 3 CDRs underlined |
| 5 | 4F11 | Tigit | Mouse VH sequence, with 3 CDRs underlined |
| 6 | | | Humanized VH sequence, with 3 CDRs underlined |
| 7 | | | Mouse VL sequence, with 3 CDRs underlined |
| 8 | | | Humanized VL sequence, with 3 CDRs underlined |
| 9 | 3G8 | CD3 | Mouse VH sequence, with 3 CDRs underlined |
| 10 | | | Humanized VH sequence, with 3 CDRs underlined |
| 11 | | | Mouse VL sequence, with 3 CDRs underlined |
| 12 | | | Humanized VL sequence, with 3 CDRs underlined |
| 13 | 8B5 | Trop2 | Mouse VH sequence, with 3 CDRs underlined |
| 14 | | | Humanized VH sequence, with 3 CDRs underlined |
| 15 | | | Mouse VL sequence, with 3 CDRs underlined |
| 16 | | | Humanized VL sequence, with 3 CDRs underlined |
| 17 | 1C8 | PD1 | Mouse VH sequence, with 3 CDRs underlined |
| 18 | | | Humanized VH sequence, with 3 CDRs underlined |
| 19 | | | Mouse VL sequence, with 3 CDRs underlined |
| 20 | | | Humanized VL sequence, with 3 CDRs underlined |
| 21 | ARB202 | CDH17/CD3 | Heavy chain |
| 22 | | | Light chain |
| 23 | ARB203 | Trop2/CD3 | Heavy chain |
| 24 | | | Light chain |
| 25 | ARB204 | CDH17/PD1 | Heavy chain |
| 26 | | | Light chain |
| 27 | ARBT01 | CDH17/CD3/PDL1 | Monomer/Chain 1 |
| 28 | | | Monomer/Chain 2 |
| 29 | U1scFv | CD3 | Humanized scFv sequence |
| 30 | h3G8v1 | CD3 | Humanized scFv sequence |
| 31 | h3G8v2 | CD3 | Humanized scFv sequence |
| 32 | h3G8v3 | CD3 | Humanized scFv sequence |
| 33 | h8B5- | Trop2/CD3 | Heavy chain |
| | h3G8v1 | | |
| 34 | h8B5- | Trop2/CD3 | Heavy chain |
| | h3G8v2 | | |
| 35 | h8B5- | Trop2/CD3 | Heavy chain |
| | h3G8v3 | | |
| 36 | 2A6 | PDL1 | Anti-PDL1 CDR-H1 sequence |
| 37 | | | Anti-PDL1 CDR-H2 sequence |
| 38 | | | Anti-PDL1 CDR-H3 sequence |
| 39 | | | Anti-PDL1 CDR-L1 sequence |
| 40 | | | Anti-PDL1 CDR-L2 sequence |
| 41 | | | Anti-PDL1 CDR-L3 sequence |
| 42 | 4F11 | Tigit | Anti-Tigit CDR-H1 sequence |
| 43 | | | Anti-Tigit CDR-H2 sequence |
| 44 | | | Anti-Tigit CDR-H3 sequence |
| 45 | | | Anti-Tigit CDR-L1 sequence |
| 46 | | | Anti-Tigit CDR-L2 sequence |
| 47 | | | Anti-Tigit CDR-L3 sequence |
| 48 | 3G8 | CD3 | Anti-CD3 CDR-H1 sequence |
| 49 | | | Anti-CD3 CDR-H2 sequence |
| 50 | | | Anti-CD3 CDR-H3 sequence |
| 51 | | | Anti-CD3 CDR-L1 sequence |
| 52 | | | Anti-CD3 CDR-L2 sequence |
| 53 | | | Anti-CD3 CDR-L3 sequence |
| 54 | 8B5 | Trop2 | Anti-Trop2 CDR-H1 sequence |
| 55 | | | Anti-Trop2 CDR-H2 sequence |
| 56 | | | Anti-Trop2 CDR-H3 sequence |
| 57 | | | Anti-Trop2 CDR-L1 sequence |
| 58 | | | Anti-Trop2 CDR-L2 sequence |
| 59 | | | Anti-Trop2 CDR-L3 sequence |
| 60 | 1C8 | PD1 | Anti-PD1 CDR-H1 sequence |
| 61 | | | Anti-PD1 CDR-H2 sequence |
| 62 | | | Anti-PD1 CDR-H3 sequence |
| 63 | | | Anti-PD1 CDR-L1 sequence |
| 64 | | | Anti-PD1 CDR-L2 sequence |
| 65 | | | Anti-PD1 CDR-L3 sequence |
| 66 | 10C12 | CDH17 | Humanized VH sequence, with 3 CDRs underlined |
| 67 | | | Humanized VL sequence, with 3 CDRs underlined |
| 68 | 10C12 | CDH17 | Anti-CDH17 CDR-H1 sequence |
| 69 | | | Anti-CDH17 CDR-H2 sequence |
| 70 | | | Anti-CDH17 CDR-H3 sequence |
| 71 | | | Anti-CDH17 CDR-L1 sequence |
| 72 | | | Anti-CDH17 CDR-L2 sequence |
| 73 | | | Anti-CDH17 CDR-L3 sequence |

### Sequence Listing

>Sequence ID NO 1: Anti-PDL1 (clone 2A6), Mouse VH sequence
QVQLQQSGAELVKPGASVKLSCKASGYTFTEYTLQWVKQRSGQGLEWIGWFFPGSGSIKYNEKFKDKATLT
ADKSSNTVYMELSRLTSEDSAVYFCARHERGFAYWGQGTLVTVST
>Sequence ID NO 2: Anti-PDL1 (clone 2A6), Humanized VH sequence
QVQLVQSGAEVKKPGASVKVSCKASGYTFTEYTMHWVRQAPGQRLEWMGWFFPGSGSIKYSQKFQGRVT
ITRDTSASTAYMELSSLRSEDTAVYYCARHERGFAYWGQGTLVTVSS
>Sequence ID NO 3: Anti-PDL1 (clone 2A6), Mouse VL sequence
QIVLTQSPAIMSASLGEEITLTCSASSSVSYMHWYQQKSGTSPKLLIYSTSNLASGVPSRFSGSGSGTFYSLTISS
VEAEDAADYHCHQWSSYPWTFGGGTKLEIKR
>Sequence ID NO 4: Anti-PDL1 (clone 2A6), Humanized VL sequence
DIVLTQSPASLAVSPGQRATITCRASASSSVSYIHWYQQKPGQPPKLLIYSTSNKDTGVPARFSGSGSGTDFTL
TINPVEANDTANYYCHQWSSYPWTFGQGTKVEIK
>Sequence ID NO 5: Anti-Tigit (clone 4F11), Mouse VH sequence
EVQLQQSGPELVKPGASVKISCKTSGYTFTEYTIHWVKQSHGKSLEWIGGINPNNGGTSYNQKFKGKATLTV
DKSSSTPYMELRSLTSEDSAVYYCARLVLRYWYFDVWGAGTTVTVSS
>Sequence ID NO 6: Anti-Tigit (clone 4F11), Humanized VH sequence
QVQLVQSGAEVKKPGASVKVSCKVSGYTFTEYTMHWVRQAPGKGLEWMGGINPNNGGTIYAQKFQGRVT
MTEDTSTDTAYMELSSLRSEDTAVYYCARLVLRYWYFDVWGRGTLVTVSS
>Sequence ID NO 7: Anti-Tigit (clone 4F11), Mouse VL sequence
QIVLTQSPAIMSASPGEKVTMTCSASSNVIYMHWYQQKSGTSPKRWLYDTSKLASGVPARFSGSGSGTSYSL
TISSMEAEDAATYYCQQWSSNPLTFGAGTKLELK
>Sequence ID NO 8: Anti-Tigit (clone 4F11), Humanized VL sequence
DIQMTQSPSSLSASVGDRVTITCRASASSNVIYLAWYQQKPGKAPKLLLYDTSRLESGVPSRFSGSGSGTDYTL
TISSLQPEDFATYYCQQWSSNPLTFGGGTKVEIK
>Sequence ID NO 9: Anti-CD3 (clone 3G8), Mouse VH sequence
QVQLQQSGPELVKPGTSVRISCKASGYTFTSYYIHWVKQRPGQGLEWIGWIYPENVNTKYNEKFKGRATLTA
DKSSNTAYMQLSSLTSEDSAVYFCARDSYGSYFFDYWGQGTTLTVSS
>Sequence ID NO 10: Anti-CD3 (clone 3G8), Humanized VH sequence
QVQLVQSGAEVKKPGASVKVSCKASGYTFTSYYMHWVRQAPGQGLEWMGWIYPENVNTNYAQKFQGRV
TMTRDTSISTAYMELSRLRSDDTAVYYCARDSYGSYFFDYWGQGTLVTVSS
>Sequence ID NO 11: Anti-CD3 (clone 3G8), Mouse VL sequence
DIVMSQSPSSLAVSAGEKVTMSCKSSQSLLNSRTRKNYLAWYQQKPGQSPKLLIYWASTRESGVPDRFTGSG
SGTDFTLTISSVQAEDLAMYYCTQSFILRTFGGGTKLEIK
>Sequence ID NO 12: Anti-CD3 (clone 3G8), Humanized VL sequence
DIVMTQSPDSLAVSLGERATINCKSSQSLLNSRTRKNYLAWYQQKPGQPPKLLIYWASTRESGVPDRFSGSGS
GTDFTLTISSLQAEDVAVYYCTQSFILRTFGQGTKVEIK
>Sequence ID NO 13: Anti-Trop2 (clone 8B5), Mouse VH sequence
EVKLVESGGGLVKPGGSLKLSCAASGFTFSTYTMSWVRQTPAKSLEWVATINSDGYNIYYSDSMKGRFTISRD
NARYTLYLQMSSLRSEDTAMYYCVRCSYYSYDYFDYWGQGTTLTVSS
>Sequence ID NO 14: Anti-Trop2 (clone 8B5), Humanized VH sequence
EVQLVESGGGLVQPGGSLRLSCAASGFTFSTYTMSWVRQTPAKGLVWVSTINSDGYNIYYSDSMKGRFTISR
DNAKYTLYLQMNSLRAEDTAMYYCARCSYYSYDYFDYWGQGTLVTVSS
>Sequence ID NO 15: Anti-Trop2 (clone 8B5), Mouse VL sequence
DIVLTQSPASLSASVGETVTITCRASENIDNYLAWYQQKQGKSPQLLVYAATNLADGMPSRFSGSGSGTQYSL
KINSLQSEDVARYFCQHYYSNQLTFGAGTKLELK
>Sequence ID NO 16: Anti-Trop2 (clone 8B5), Humanized VL sequence
DIQMTQSPSSLSASVGDRVTITCRASENIDNYLAWYQQKQGKVPKLLIYAATNLADGMPSRFSGSGSGTDFT
LTISSLQPEDVATYYCQHYYSNQLTFGQGTKLEIK
>Sequence ID NO 17: Anti-PD1 (clone 1C8), Mouse VH sequence
EVILVESGGDLVKPGGSLKLSCAASGFTFSRSTMSWVRQTPEKRLEWVATISGGGLNTYFPDSVKGRFTISRD
NAKNTLYLHMSSLRSEDTAVYYCTRNSNSYYFALDYWGQGTSVTVSS
>Sequence ID NO 18: Anti-PD1 (clone 1C8), Humanized VH sequence
EVQLVESGGGLVQPGGSLRLSCAASGFTFSRSTMSWVRQAPGKGLEWVATISGGGLNTYFPDSVKGRFTISR
DNSKNTLYLQMNSLRAEDTAVYYCTRNSNSYYFALDYWGQGTLVTVSS
>Sequence ID NO 19: Anti-PD1 (clone 1C8), Mouse VL sequence
DIVLTQSPASLAVSLGQRATISCRASESVDNSGVSFMNWFQQKPGQSPKLLIYIASNQVSGVPARFSGSGSGT
DFSLNIHPMEEDDTAMYFCQQIKEVPWTFGGGTKLEIK
>Sequence ID NO 20: Anti-PD1 (clone 1C8), Humanized VL sequence
EIVLTQSPATLSLSPGERATLSCRASESVDNSGVSFMNWYQQKPGQAPRLLIYIASNQVSGIPARFSGSGSGT
DFTLTISSLEPEDFAVYYCQQIKEVPWTFGGGTKVEIK
>Sequence ID NO 21: ARB202, heavy chain
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSAASTKGPS
VFPLAPSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLGKPAGGGGSEVQLVESGGGLVQPGGSLRLSCAASGYSFTGYT
MNWVRQAPGKGLEWVALINPYKGVSTYNQKFKDRFTISVDKSKNTAYLQMNSLRAEDTAVYYCARSGYYG
DSDWYFDVWGQGTLVTVSSGGGGSGGGGSGGGGSDIQMTQSPSSLSASVGDRVTITCRASQDIRNYLNW
YQQKPGKAPKLLIYYTSRLESGVPSRFSGSGSGTDYTLTISSLQPEDFATYYCQQGNTLPWTFGQGTKVEIK
>Sequence ID NO 22: ARB202, light chain
MRLPAQLLGLLMLWVSGSSGDIQMTQSPSSLSASVGDRVTITCRASQDISGYLNWLQQKPGGAIKRLIYTTS
TLDSGVPKRFSGSGSGTDFTLTISSLQSEDFATYYCLQYASSPFTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVYACEVTHQG
LSSPVTKSFNRGEC
>Sequence ID NO 23: ARB203, heavy chain
MEFGLSWVFLVALLRGVQCEVQLVESGGGLVQPGGSLRLSCAASGFTFSTYTMSWVRQTPAKGLVWVSTI
NSDGYNIYYSDSMKGRFTISRDNAKYTLYLQMNSLRAEDTAMYYCARCSYYSYDYFDYWGQGTLVTVSSAST
KGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGKPAGGGGSEVQLVESGGGLVQPGGSLRLSCAASGYS
FTGYTMNWVRQAPGKGLEWVALINPYKGVSTYNQKFKDRFTISVDKSKNTAYLQMNSLRAEDTAVYYCARS
GYYGDSDWYFDVWGQGTLVTVSSGGGGSGGGGSGGGGSDIQMTQSPSSLSASVGDRVTITCRASQDIRN
YLNWYQQKPGKAPKLLIYYTSRLESGVPSRFSGSGSGTDYTLTISSLQPEDFATYYCQQGNTLPWTFGQGTKV
EIK
>Sequence ID NO 24: ARB203, light chain
MRLPAQLLGLLMLWVSGSSGDIQMTQSPSSLSASVGDRVTITCRASENIDNYLAWYQQKQGKVPKLLIYAAT
NLADGMPSRFSGSGSGTDFTLTISSLQPEDVATYYCQHYYSNQLTFGQGTKLEIKRTVAAPSVFIFPPSDEQLK
SGTASVVCLLNNFYPREAKVQWKVDNALQSGNSQESVTEQDSKDSTYSLSSTLTLSKADYEKHKVYACEVTH
QGLSSPVTKSFNRGEC
>Sequence ID NO 25: ARB204, heavy chain
MEFGLSWVFLVALLRGVQCEVQLVESGGGLVQPGGSLRLSCAASGFTFSSYAMSWVRQTPGKGLEWVAVI
DSNGGSTYYPDTVKDRFTISRDNSKNTLYLQMNSLRAEDTAVYYCSSYTNLGAYWGQGTLVTVSAASTKGPS
VFPLAPSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKDTLMISRTPEVTCVVVDVSQEDPE
VQFNWYVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLTVDKSR
WQEGNVFSCSVMHEALHNHYTQKSLSLSLGKPAGGGGSEVQLVESGGGLVQPGGSLRLSCAASGFTFSRST
MSWVRQAPGKGLEWVATISGGGLNTYFPDSVKGRFTISRDNSKNTLYLQMNSLRAEDTAVYYCTRNSNSYY
FALDYWGQGTLVTVSSGGGGSGGGGSGGGGSEIVLTQSPATLSLSPGERATLSCRASESVDNSGVSFMNWY
QQKPGQAPRLLIYIASNQVSGIPARFSGSGSGTDFTLTISSLEPEDFAVYYCQQIKEVPWTFGGGTKVEIK
>Sequence ID NO 26: ARB204, light chain
MRLPAQLLGLLMLWVSGSSGDIQMTQSPSSLSASVGDRVTITCRASQDISGYLNWLQQKPGGAIKRLIYTTS
TLDSGVPKRFSGSGSGTDFTLTISSLQSEDFATYYCLQYASSPFTFGGGTKVEIKRTVAAPSVFIFPPSDEQLKSG
TASVVCLLNNFYPREAKVQWKVDNALQSGNSQESVTEQDSKDSTYSLSSTLTLSKADYEKHKVYACEVTHQG
LSSPVTKSFNRGEC
>Sequence ID NO 27: ARBT01, chain 1
GTATMRLPAQLLGLLMLWVSGSSGDIQMTQSPSSLSASVGDRVTITCQASQDISNYLNWYQQKPGKAPKLL
IYYTSRLESGVPSRFSGSGSGTDYTLTISSLQPEDFATYYCQQGNTLPWTFGQGTKVEIKGAPGGGGTHTCPP
CPAPEFLGGPSVFLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTKNQVSLSCAVKGFY
PSDIAVEWESNGQPENNYKTTPPVLDSDGSF
FLVSKLTVDKSRWQEGNVFSCSVMHEALHNHYTQKSLSLSLSPGKPAGGGGSEVQLLESGGGLVQPGGSLRL
SCAASGFTFSSYAMSWVRQAPGKGLEWVAVIDSNGGSTYYPDTVKDRFTISRDNSKNTLYLQMNSLRAEDT
AVYYCSSYTNLGAYWGQGTLVTVSAGGGGSGGGGSGGGGSDIQMTQSPSSLSASVGDRVTITCRASQDISG
YLNWLQQKPGKAIKRLIYTTSTLDSGVPSRFSGSGSGTDFTLTISSLQPEDFATYYCLQYASSPFTFGGGTKVEIK
>Sequence ID NO 28: ARBT01, chain 2
MEFGLSWVFLVALLRGVQCQVQLVQSGAEVKKPGASVKVSCKASGYTFTEYTMHWVRQAPGQRLEWMG
WFFPGSGSIKYSQKFQGRVTITRDTSASTAYMELSSLRSEDTAVYYCARHERGFAYWGQGTLVTVSSGGGGS
GGGGSGGGGSDIVLTQSPASLAVSPGQRATITCRASASSSVSYIHWYQQKPGQPPKLLIYSTSNKDTGVPARF
SGSGSGTDFTLTINPVEANDTANYYCHQWSSYPWTFGQGTKVEIKGGGGSQVQLVQSGAEVKKPGASVKV
SCKASGYSFTDYTMNWVRQAPGQGLEWMGVINPNHGISSYAQKFQGRVTMTRDTSTSTVYMELSSLRSED
TAVYYCVRRKISYDYDEGYAMDYWGQGTLVTVSSGAPGGGGTHTCPPCPAPEFLGGPSVFLFPPKPKDTLMI
SRTPEVTCVVVDVSQEDPEVQFNWYVDGVEVHNAKTKPREEQFNSTYRVVSVLTVLHQDWLNGKEYKCKV
SNKGLPSSIEKTISKAKGQPREPQVYTLPPSQEEMTKNQVSLWCLVKGFYPSDIAVEWESNGQPENNYKTTP
PVLDSDGSFFLYSKLTVDKSRWQEGNVFSCSVMHEALHNHYTQKSLSLSLGK
>Sequence ID NO 29: UCHT1, humanized scFv
EVQLVESGGGLVQPGGSLRLSCAASGYSFTGYTMNWVRQAPGKGLEWVALINPYKGVSTYNQKFKDRFTIS
VDKSKNTAYLQMNSLRAEDTAVYYCARSGYYGDSDWYFDVWGQGTLVTVSSGGGGSGGGGSGGGGSDI
QMTQSPSSLSASVGDRVTITCRASQDIRNYLNWYQQKPGKAPKLLIYYTSRLESGVPSRFSGSGSGTDYTLTIS
SLQPEDFATYYCQQGNTLPWTFGQGTKVEIK
>Sequence ID NO 30: h3G8v1, humanized scFv
QVQLVQSGAEVKKPGASVKVSCKASGYTFTSYYMHWVRQAPGQGLEWMGWIYPENVNTNYAQKFQGRV
TMTRDTSISTAYMELSRLRSDDTAVYYCARDSYGSYFFDYWGQGTLVTVSSGGGGSGGGGSGGGGSDIVM
TQSPDSLAVSLGERATINCKSSQSLLNSRTRKNYLAWYQQKPGQPPKLLIYWASTRESGVPDRFSGSGSGTDF
TLTISSLQAEDVAVYYCTQSFILRTFGQGTKVEIK
>Sequence ID NO 31: h3G8v2, humanized scFv
DIVMTQSPDSLAVSLGERATINCKSSQSLLNSRTRKNYLAWYQQKPGQPPKLLIYWASTRESGVPDRFSGSGS
GTDFTLTISSLQAEDVAVYYCTQSFILRTFGQGTKVEIKGGGGSGGGGSGGGGSQVQLVQSGAEVKKPGASV
KVSCKASGYTFTSYYMHWVRQAPGQGLEWMGWIYPENVNTNYAQKFQGRVTMTRDTSISTAYMELSRLR
SDDTAVYYCARDSYGSYFFDYWGQGTLVTVSS
>Sequence ID NO 32: h3G8v3, humanized scFv
QVQLVQSGAEVKKPGASVKVSCKASGYTFTSYYMHWVRQAPGQGLEWMGWIYPENVNTNYAQKFQGRV
TMTRDTSISTAYMELSRLRSDDTAVYYCARDSYGSYFFDYWGQGTLVTVSSGGGGSGGGGSGGGGSGGGG
SGGGGSGGGGSDIVMTQSPDSLAVSLGERATINCKSSQSLLNSRTRKNYLAWYQQKPGQPPKLLIYWASTRE
SGVPDRFSGSGSGTDFTLTISSLQAEDVAVYYCTQSFILRTFGQGTKVEIK
>Sequence ID NO 33: h8B5-h3G8v1, heavy chain
MEFGLSWVFLVALLRGVQCEVQLVESGGGLVQPGGSLRLSCAASGFTFSTYTMSWVRQTPAKGLVWVSTI
NSDGYNIYYSDSMKGRFTISRDNAKYTLYLQMNSLRAEDTAMYYCARCSYYSYDYFDYWGQGTLVTVSSAST
KGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGKPAGGGGSQVQLVQSGAEVKKPGASVKVSCKASGY
TFTSYYMHWVRQAPGQGLEWMGWIYPENVNTNYAQKFQGRVTMTRDTSISTAYMELSRLRSDDTAVYYC
ARDSYGSYFFDYWGQGTLVTVSSGGGGSGGGGSGGGGSDIVMTQSPDSLAVSLGERATINCKSSQSLLNSR
TRKNYLAWYQQKPGQPPKLLIYWASTRESGVPDRFSGSGSGTDFTLTISSLQAEDVAVYYCTQSFILRTFGQG
TKVEIK
>Sequence ID NO 34: h8B5-h3G8v2, heavy chain
MEFGLSWVFLVALLRGVQCEVQLVESGGGLVQPGGSLRLSCAASGFTFSTYTMSWVRQTPAKGLVWVSTI
NSDGYNIYYSDSMKGRFTISRDNAKYTLYLQMNSLRAEDTAMYYCARCSYYSYDYFDYWGQGTLVTVSSAST
KGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGKPAGGGGSDIVMTQSPDSLAVSLGERATINCKSSQS
LLNSRTRKNYLAWYQQKPGQPPKLLIYWASTRESGVPDRFSGSGSGTDFTLTISSLQAEDVAVYYCTQSFILRT
FGQGTKVEIKGGGGSGGGGSGGGGSQVQLVQSGAEVKKPGASVKVSCKASGYTFTSYYMHWVRQAPGQ
GLEWMGWIYPENVNTNYAQKFQGRVTMTRDTSISTAYMELSRLRSDDTAVYYCARDSYGSYFFDYWGQG
TLVTVSS
>Sequence ID NO 35: h8B5-h3G8v3, heavy chain
MEFGLSWVFLVALLRGVQCEVQLVESGGGLVQPGGSLRLSCAASGFTFSTYTMSWVRQTPAKGLVWVSTI
NSDGYNIYYSDSMKGRFTISRDNAKYTLYLQMNSLRAEDTAMYYCARCSYYSYDYFDYWGQGTLVTVSSAST
KGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGKPAGGGGSQVQLVQSGAEVKKPGASVKVSCKASGY
TFTSYYMHWVRQAPGQGLEWMGWIYPENVNTNYAQKFQGRVTMTRDTSISTAYMELSRLRSDDTAVYYC
ARDSYGSYFFDYWGQGTLVTVSSGGGGSGGGGSGGGGSGGGGSGGGGSGGGGSDIVMTQSPDSLAVSL
GERATINCKSSQSLLNSRTRKNYLAWYQQKPGQPPKLLIYWASTRESGVPDRFSGSGSGTDFTLTISSLQAED
VAVYYCTQSFILRTFGQGTKVEIK
>Sequence ID NO 36: Anti-PDL1 (clone 2A6) CDR-H1 sequence
GYTFTEYT
>Sequence ID NO 37: Anti-PDL1 (clone 2A6) CDR-H2 sequence
FFPGSGSI
>Sequence ID NO 38: Anti-PDL1 (clone 2A6) CDR-H3 sequence
ARHERGFAY
>Sequence ID NO 39: Anti-PDL1 (clone 2A6) CDR-L1 sequence
ASSSVSY
>Sequence ID NO 40: Anti-PDL1 (clone 2A6) CDR-L2 sequence
STS
>Sequence ID NO 41: Anti-PDL1 (clone 2A6) CDR-L3 sequence
HQWSSYPWT
>Sequence ID NO 42: Anti-Tigit (clone 4F11) CDR-H1 sequence
GYTFTEYT
>Sequence ID NO 43: Anti-Tigit (clone 4F11) CDR-H2 sequence
INPNNGGT
>Sequence ID NO 44: Anti-Tigit (clone 4F11) CDR-H3 sequence
ARLVLRYWYFDV
>Sequence ID NO 45: Anti-Tigit (clone 4F11) CDR-L1 sequence
ASSNVIY
>Sequence ID NO 46: Anti-Tigit (clone 4F11) CDR-L2 sequence
DTS
>Sequence ID NO 47: Anti-Tigit (clone 4F11) CDR-L3 sequence
QQWSSNPLT
>Sequence ID NO 48: Anti-CD3 (clone 3G8) CDR-H1 sequence
GYTFTSYY
>Sequence ID NO 49: Anti-CD3 (clone 3G8) CDR-H2 sequence
IYPENVNT
>Sequence ID NO 50: Anti-CD3 (clone 3G8) CDR-H3 sequence ARDSYGSYFFDY
>Sequence ID NO 51: Anti-CD3 (clone 3G8) CDR-L1 sequence QSLLNSRTRKNY
>Sequence ID NO 52: Anti-CD3 (clone 3G8) CDR-L2 sequence
WAS
>Sequence ID NO 53: Anti-CD3 (clone 3G8) CDR-L3 sequence TQSFILRT
>Sequence ID NO 54: Anti-Trop2 (clone 8B5) CDR-H1 sequence
GFTFSTYT
>Sequence ID NO 55: Anti-Trop2 (clone 8B5) CDR-H2 sequence
INSDGYNI
>Sequence ID NO 56: Anti-Trop2 (clone 8B5) CDR-H3 sequence ARCSYYSYDYFDY
>Sequence ID NO 57: Anti-Trop2 (clone 8B5) CDR-L1 sequence ENIDNY
>Sequence ID NO 58: Anti-Trop2 (clone 8B5) CDR-L2 sequence
AAT
>Sequence ID NO 59: Anti-Trop2 (clone 8B5) CDR-L3 sequence
QHYYSNQLT
>Sequence ID NO 60: Anti-PD1 (clone 1C8) CDR-H1 sequence
GFTFSRST
>Sequence ID NO 61: Anti-PD1 (clone 1C8) CDR-H2 sequence
ISGGGLNTYFPDSVKGRFTI
>Sequence ID NO 62: Anti-PD1 (clone 1C8) CDR-H3 sequence
TRNSNSYYFALDY
>Sequence ID NO 63: Anti-PD1 (clone 1C8) CDR-L1 sequence
ESVDNSGVSF
>Sequence ID NO 64: Anti-PD1 (clone 1C8) CDR-L2 sequence
IAS
>Sequence ID NO 65: Anti-PD1 (clone 1C8) CDR-L3 sequence
QQIKEVPWT
>Sequence ID NO 66: Anti-CDH17 (clone 10C12), Humanized VH sequence
EVQLVESGGGLVQPGGSLRLSCAASGFTFSSYAMSWVRQTPGKGLEWVAVIDSNGGSTYYPDTVKDRFTISR
DNSKNTLYLQMNSLRAEDTAVYYCSSYTNLGAYWGQGTLVTVSA
>Sequence ID NO 67: Anti-CDH17 (clone 10C12), Humanized VL sequence
DIQMTQSPSSLSASVGDRVTITCRASQDISGYLNWLQQKPGGAIKRLIYTTSTLDSGVPKRFSGSGSGTDFTLTI
SSLQSEDFATYYCLQYASSPFTFGGGTKVEIK
>Sequence ID NO 68: Anti-CDH17 (clone 10C12), CDR-H1 sequence
GFTFSSYA
>Sequence ID NO 69: Anti-CDH17 (clone 10C12), CDR-H2 sequence
IDSNGGST
>Sequence ID NO 70: Anti-CDH17 (clone 10C12), CDR-H3 sequence
SSYTNLGAY
>Sequence ID NO 71: Anti-CDH17 (clone 10C12), CDR-L1 sequence
QDISGY
>Sequence ID NO 72: Anti-CDH17 (clone 10C12), CDR-L2 sequence
TTS
>Sequence ID NO 73: Anti-CDH17 (clone 10C12), CDR-L3 sequence
LQYASSPFT

What is claimed is:

1. A bispecific antibody, comprising IgG domains having heavy chains and light chains, and two scFv domains being connected to C terminal of the heavy chains, wherein the IgG domains comprises Fab regions having the binding specificity to a first antigen selected from Trop2 and CDH17, wherein the scFv domains have the binding specificity to a second antigen selected from CD3 and PD1.

2. The bispecific antibody of claim 1, wherein the Fab domain comprises an amino acid sequence having at least 98% of sequence identity to an amino acid sequence selected from SEQ ID NO: 13, 14, 15, 16, 66, and 67.

3. The bispecific antibody of claim 1, wherein the scFv domain comprises an amino acid sequence having at least 98% of sequence identity to an amino acid sequence selected from SEQ ID NO: 29, 30, 31, and 32.

4. The bispecific antibody of claim 1, comprising,
3 heavy chain complementary determining regions (CDRs) of SEQ ID NO: 54, 55, 56 and 3 light chain CDRs of SEQ ID NO: 57, 58, 59, or
3 heavy chain CDRs of SEQ ID NO: 68, 69, 70 and 3 light chain CDRs of SEQ ID NO: 71, 72, 73.

5. The bispecific antibody of claim 1, comprising,
3 heavy chain CDRs of SEQ ID NO: 48, 49, 50 and 3 light chain CDRs of SEQ ID NO: 51, 52, 53, or
3 heavy chain CDRs of SEQ ID NO: 60, 61, 62 and 3 light chain CDRs of SEQ ID NO: 63, 64, 65.

6. The bispecific antibody of claim 1, wherein the first antigen is Trop2, and the second antigen is CD3.

7. The bispecific antibody of claim 6, comprising an amino acid sequence having at least 98% of sequence identity to an amino acid sequence selected from SEQ ID NO: 23, 24, 33, 34, 35.

8. The bispecific antibody of claim 1, wherein the first antigen is CDH17, and the second antigen is PD1.

9. The bispecific antibody of claim 8, comprising an amino acid sequence having at least 98% of sequence identity to an amino acid sequence selected from SEQ ID NO: 25, 26.

10. The bispecific antibody of claim 1, wherein the antibody is a humanized antibody, a chimeric antibody, or a recombinant antibody, or an isolated monoclonal antibody.

11. An isolated nucleic acid sequence encoding the bispecific antibody of claim 1.

12. An expression vector comprising the isolated nucleic acid sequences of claim 11.

13. A host cell comprising the isolated nucleic acid sequence of claim 11.

14. A method for producing the bispecific antibody of claim 1, comprising culturing the host cell of claim 13, so that the antibody is produced.

15. A method for treating or preventing a cancer, an autoimmune disease, or an infectious disease in a subject, said method comprising administering to the subject a pharmaceutical composition comprising a bispecific antibody of claim 1.

16. A method of treating a subject with a cancer, comprising administering to the subject an effective amount of the bispecific antibody of claim 1.

17. The method of claim 28, further comprising co-administering an effective amount of a therapeutic agent.

18. The method of claim 28, wherein the therapeutic agent comprises an antibody, a chemotherapy agent, an enzyme, or a combination thereof.
